(19) United States
(12) Patent Application Publication
(10) Pub. No.: US 2026/0198818 A1
(43) Pub. Date: Jul. 16, 2026

(54) Dried Blood Spot Collection Card Having Reduced Material Space for Reduced Blood Sampling

(71) Applicant: Blackfly Investments, LLC, dba Molecular Testing Labs, Vancouver, WA (US)

(72) Inventors: Mariko Nakano, Vancouver, WA (US); Ryan Baran-Maguire, Portland, OR (US)

(21) Appl. No.: 19/020,544

(22) Filed: Jan. 14, 2025

(51) Int. Cl. A61B 5/15

(52) U.S. Cl. CPC A61B 5/150358; A61B 5/150022; A61B 2505/07

(57) ABSTRACT

A dried blood spot collection (DBS) card that assists with correct and efficient collection of capillary blood using a dried blood spot collection (DBS) collection device. The DBS card may comprise a rectangular shape configured to be held in a DBS collection device in a desired orientation for blood sample collection. The DBS card comprises target discs aligned with the collection apertures on the DBS collection device. The patient will be able to direct drops of capillary blood to pass through the top holes and then blood will travel into the DBS card at precise locations so as to ensure proper saturation while minimizing risk of outside contaminants and/or cross-contamination between target spots. Further, the target spots are physically surrounded by voids separated by bridges such that the patient can verify saturation level by looking at the saturation of the central target disc.

### BACKGROUND

As techniques and methodologies for medical testing and diagnostics advance, more and more consumers are turning to remote sample collection using “at-home” test kits that may involve collecting a blood sample using a Dried Blood Spot (DBS) card having the means to collect, store, and ship blood samples from patients to remote diagnostics center for further testing and analysis. In a typical procedure, a patient will induce a blood flow from a finger by pricking the finger allowing blood to flow for enough time to drip onto a dedicated collection medium, such as a DBS card. As is known in the profession, DBS cards may have a typical form factor wherein a patient may deposit up to five blood samples, or more, in dedicated sample areas and then, after allowing time to dry, package the entire DBS card for mailing or transit to a remote testing facility. Results of testing or diagnostics may then be communicated to the patient using standard confidentiality protocols.

However, the standard DBS card form factor is prone to problems such as contamination or spoiled samples and often require a relatively large amount of collected blood for proper sampling and target saturation. That is, as the portion of the DBS card that collects the blood sample is open to human touching and foreign objects during the collection and transit procedures, the blood sample itself may be compromised to the point of causing unreliable testing results. Further, as the DBS card typically has several “spots” or “targets” available for blood collection, often times, a typical finger prick may not provide enough blood to fill all the spots as enough blood is needed in each spot to thoroughly saturate the target area. This may prompt the patient to collect from another finger after some time. If a blood collection spot dries before being completed or has additional samples to it after some drying has occurred, the entire sample may also be compromised. As is standard with self-collection test kits, collection should be prompt and distinct for each of the typical five spots in order to maximize the chances for proper and reliable testing and diagnostics. Thus, conventional open faced DBS cards are susceptible to opportunities for failure during blood collection and transit.

### BRIEF DESCRIPTION OF THE DRAWINGS

Embodiments of the subject matter disclosed herein in accordance with the present disclosure will be described with reference to the drawings, in which:

FIG. 1 is a top-view diagram of a conventional dried blood spot collection card having large target areas;

FIG. 2 is a top-view diagram of a dried blood spot collection device for use with an objective dried blood spot collection card according to an embodiment of the subject matter disclosed herein;

FIG. 3 is a diagram view of the top side of the objective dried blood spot collection device of FIG. 2 according to an embodiment of the subject matter disclosed herein;

FIG. 4 is a top-view diagram of a dried blood spot collection card for use with an objective dried blood spot collection device having target spots with strategically removed material for enabling efficient DBS spotting according to an embodiment of the subject matter disclosed herein;

FIG. 5 is a diagram view of the objective dried blood spot collection card of FIG. 4 inside the dried blood spot collection device of FIG. 2 being used in a process for collecting capillary blood from a human according to an embodiment of the subject matter disclosed herein;

FIG. 6 is a close-up diagram view of one target of the objective dried blood spot collection card of FIG. 4 properly saturated with capillary blood from a human according to an embodiment of the subject matter disclosed herein;

FIG. 7 is a diagram view of another embodiment of an objective dried blood spot collection card according to an embodiment of the subject matter disclosed herein; and

FIG. 8 is a diagram view of yet another embodiment an objective dried blood spot collection card according to an embodiment of the subject matter disclosed herein.

Note that the same numbers are used throughout the disclosure and figures to reference like components and features.

### DETAILED DESCRIPTION

The subject matter of embodiments disclosed herein is described here with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

Embodiments will be described more fully hereinafter with reference to the accompanying drawings, which form a part hereof, and which show, by way of illustration, exemplary embodiments by which the devices described herein may be practiced. These devices may, however, be embodied in many different forms and should not be construed as limited to the embodiments set forth herein; rather, these embodiments are provided so that this disclosure will satisfy the statutory requirements and convey the scope of the subject matter to those skilled in the art.

By way of an overview, the systems and devices discussed herein may be directed to a dried blood spot (DBS) card used in conjunction with an objective DBS collection device that assists with correct and efficient collection of capillary blood onto the DBS card. The DBS card may comprise a rectangular shape configured to be held in a DBS collection device in a desired orientation for blood sample collection. The DBS card comprises target spots having quadrants of material removed and aligned with the collection apertures on the DBS collection device. The patient will be able to direct drops of capillary blood to pass through the top holes and then blood will travel into the DBS card at precise locations so as to ensure proper saturation of the isolated quadrant-style target while minimizing risk of outside contaminants and/or cross-contamination between target spots. Further, the target spots are physically separated with quadrant vacancies of the card material so as to reduce the amount of blood needed to reach proper saturation such that the patient can verify saturation level by looking.

This collection assistance device is an improvement to the collection because the device prevents the patient from rubbing a finger directly on the DBS card and prevents the patient from spotting blood on the wrong side of the DBS card. Further, the device promotes the capillary blood to be collected in manner that is centered on each target spot on the DBS card and additionally prevents for blood to be spotted in other areas of the DBS card. Further quadrant vacancies or partitions keep blood samples from saturating into adjacent blood samples on the novel DBS cards used in the system. These and other aspects of the subject matter disclosed herein are described below with respect to FIGS. 1-8.

FIG. 1 is an isometric diagram of a conventional dried blood spot (DBS) collection card 100 having a standard form factor. Dried blood spot testing (DBS) is a form of bio sampling where blood samples are blotted and dried on filter paper, such as Whatman™ 903 filter paper. The dried samples can easily be shipped to a remote analytical laboratory and analyzed using various methods. Dried blood spot specimens are collected by applying a few drops of blood, drawn by lancet (e.g., finger prick) from the finger, heel or toe, onto specially manufactured absorbent filter paper. The blood is allowed to thoroughly saturate the filter paper at five specific target circles 104a-e and is air dried for several hours. In this conventional example, the number of target spots 104a-e is five. Furthermore, the dimensions of the dried blood spot collection card 100 may be a standard form factor having a length of approximately three inches and a width of five inches, though a skilled artisan understands that this size can be any convenient dimension and the number of spots may be any number including between two and twelve spots.

Once dried, specimens may be stored in low gas-permeability plastic bags with desiccant added to reduce humidity, and may be kept at ambient temperature prior to, during, and after shipping. Once in the laboratory, technicians may separate a small disc of saturated paper (e.g., poke a hole) from the sheet using an automated or manual hole punch, dropping the separated disc into a flat-bottomed microtiter plate. The blood is eluted out from the separated disc into a phosphate buffered saline. The resultant plate containing the eluates forms the “master” from which dilutions can be made for subsequent testing.

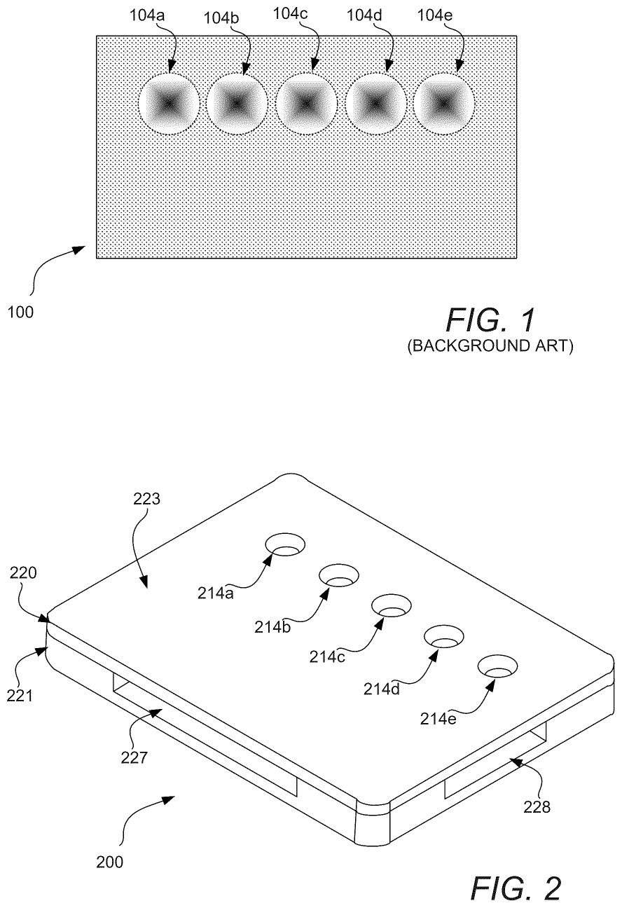

This DBS card 100 may be utilized with an objective dried blood collection device (FIG. 2) which is the subject of related U.S. patent application Ser. No. ______ and incorporated herein by reference in its entirety for all purposes.

FIG. 2 is an isometric diagram of an objective dried blood spot collection device 200 according to an embodiment of the subject matter disclosed herein. The device 200 is designed to assist with correct and efficient collection of capillary blood onto dried blood spot collection (DBS) card (400 of FIG. 4 but not shown in FIG. 2). The device 200 may comprise a body (comprising top portion 220 and bottom portion 221) having a rectangular shape configured to hold a DBS card 400 in a cavity (not shown). The DBS card 400 is held in the cavity between the top portion 220 and the bottom portion 221 of the device 200 in a desired orientation (e.g., the target circles 404a-404e of FIG. 4 are aligned with the collection apertures 214a-214e as discussed below) for blood sample collection. The top portion 220 and bottom portion 221 are separable and held together in a mating nature using mating portions (not shown) so as to allow access to and removal of the encompassed DBS card 300 when separated. The portions 220 and 221 may be made from an extruded or molded polyurethane plastic having an SPI rating of A2.

The top portion 220 includes five apertures 214a-214e in a top face 223 for assisting in collecting capillary blood from a patient. In embodiments, each aperture 214a-214e comprises a conical opening with an upper diameter (e.g., in the plane of the top face 223) that is between 0.5 to 1.5 inches. The lower diameter is slightly less to provide a conical shape to each aperture 214a-214e. The bottom portion 221 also includes a bottom face that is unable to be seen in FIG. 2.

When mated, the top portion 220 and bottom portion 221 may exhibit a first elongate ventilation opening 227 along a first direction in two sides (only the one side is shown in FIG. 2) of the overall device 200 that exposes the cavity (where the DBS card 400 is secured) to ambient air. Thus, the first ventilation opening 227 provides a means for assisting in the drying procedure after blood has been collected onto the DBS card 400. Similarly, when mated, the top portion 220 and bottom portion 221 may exhibit a second ventilation opening 228 along a second direction in two sides (only the one side is shown in FIG. 2) of the overall device 200 that exposes the cavity (where the DBS card 400 is secured) to ambient air. As such, the second ventilation opening 228 also provides additional means for assisting in the drying procedure after blood has been collected onto the DBS card 400.

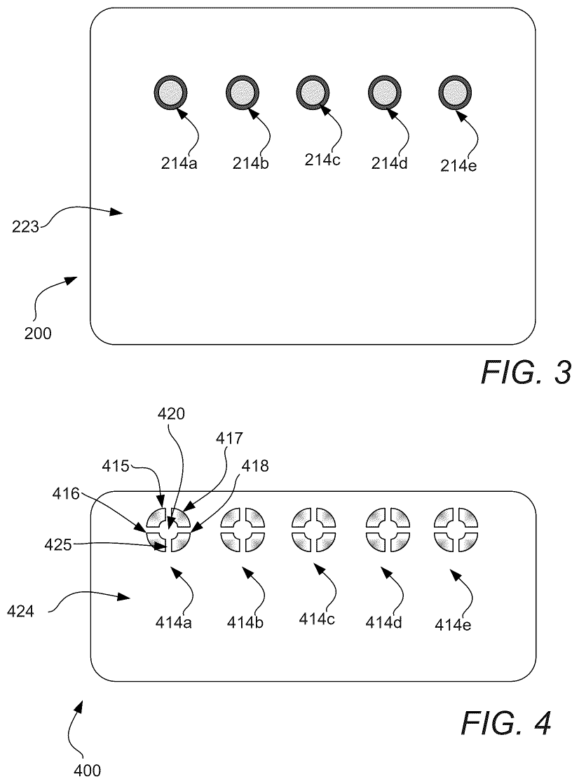

FIG. 3 is a diagram view of the top face 223 of the objective dried blood spot collection device 200 of FIG. 2 according to an embodiment of the subject matter disclosed herein. In this view, the apertures 214a-214e can be seen from a directly overhead view revealing the conical nature of the apertures 214a-214e. Thus, as blood is passed through these apertures 214a-214e, the underlying DBS card 400 may be saturated with blood as needed for the collection procedure. The device 200, as alluded to above, comprises five apertures 214a-214e on the top face 223 of the top portion 220 that are aligned above the circular target spots 404a-404e on the encompassed DBS card 400. When is use, a patient (not shown) will be able to direct drops of capillary blood to pass through the top side 223 such that blood will travel through the apertures 214a-214e to the DBS card 400 at precise locations so as to ensure proper saturation of each respective target spot 404e-404e while minimizing risk of outside contaminants and/or cross-contamination between target spots 404a-404e.

FIG. 4 is a top-view diagram of a dried blood spot collection card 400 for use with an objective dried blood spot collection device (not shown in FIG. 4) having targets with a target disc 420 surrounded by tetrapartite voids 415-418 comprising strategically removed card material for enabling efficient DBS spotting according to an embodiment of the subject matter disclosed herein. In this embodiment, the voids 415-418 are tetrapartite voids (e.g., four voids surrounding the central target disc 420). The design of the DBS card 400 having tetrapartite voids 415-418 in crescent-like shapes limits the amount of blood required to successfully saturate the outlined 6 mm target disc 420 when separated after being received at a laboratory for diagnostic testing. The unique design of the four bridges 425 connecting the 6 mm discs to the rest of the card material 424 allows the excess blood to be absorbed and pulled away from the central target disc 420. This absorption mechanism reduces drying time, while simultaneously limiting the required sample volume and providing the user with a visual representation of a complete sample deposit by spreading the blood past the bridges 425.

In this embodiment, the voids 415-418 are tetrapartite voids (e.g., four voids or relatively equal area) surrounding the central target disc 420. In other embodiments, the voids may be quinquepartite (e.g., five void areas with five bridges 425) or tripartite (e.g., three void areas with three bridges 425). The quinquepartite and tripartite embodiments are not shown in any figure but may be understood by a skilled artisan in view of the tetrapartite embodiment of FIG. 4. Further, the DBS card 400 is made of thick cellulose cardstock, with 6-mm diameter target discs 420 for blood collection. The inner diameter of the cut-out void sections 415-418 is 6-mm, while the outer diameter of the voids 415-418 is 12-mm.

The DBS card 400 of FIG. 4 with tetrapartite voids 415-418 surrounding the central target disc have numerous advantages over the conventional DBS card 100 of FIG. 1. In a first advantage, the DBS card 400 with limited absorptive volume because of the tetrapartite voids 415-418 exhibits a reduced drying time after sample collection. This is because less blood is required to establish, via visual inspection, proper saturation. Conventional DBS cards 100 require significant increases in drying time due to the greater sample volume needed to saturate a full target spot. This increase in drying time slows down the process, whether the collection is done in-home or at a medical facility, as the sample must be fully dried before shipping. Having a smaller target area 420 with empty voids 415-418 surrounding the target makes blood collection more efficient through faster drying times and less blood required.

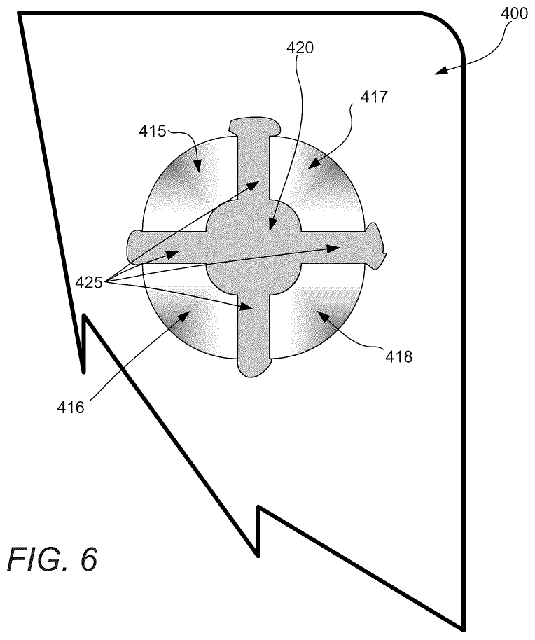

Additionally, a second advantage is that a visual inspection of the DBS card 400 is more readily achieved as excess blood is wicked away from the central target disc 420 by the bridges 425 to show that saturation of the central target disc 420 has been achieved. Conventional systems use DBS cards with constant cross-sectional areas, meaning the conventional cards do not have voids or bridges. These designs lead to issues with inconsistent blood collection such as over-saturation, double-spotting, and under-saturation. These concepts are illustrated below with respect to FIG. 6.

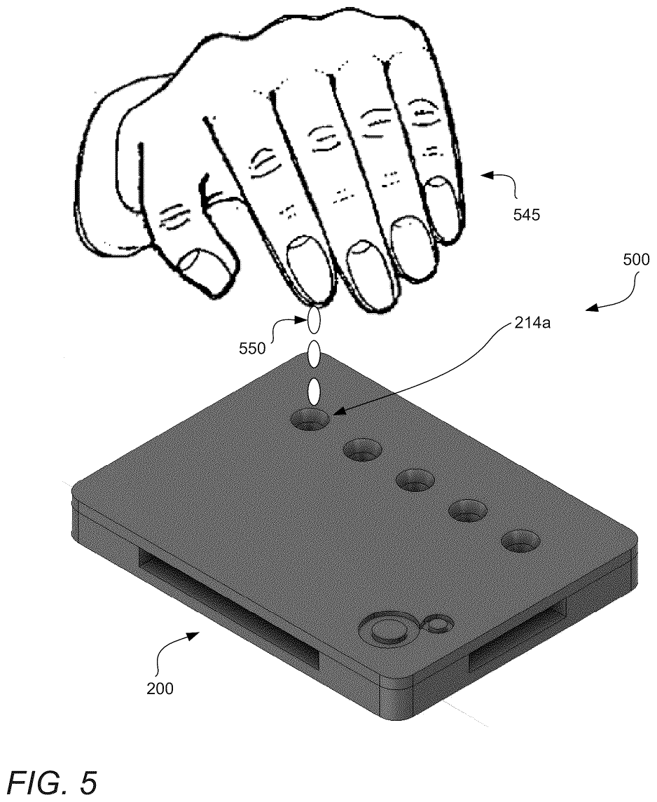

FIG. 5 is a diagram view of a system 500 comprising the objective dried blood spot collection device 200 of FIG. 2 being used in a process for collecting capillary blood 550 from a human 545 according to an embodiment of the subject matter disclosed herein. In this view, one can see a human hand 545 of a human patient dripping blood 550 (e.g., after being lanced) such that capillary blood 550 may drip down into, in this view, a first collection aperture 214a of the device 200. As the (unseen) DBS collection card 400 becomes saturated, the patient may move the lanced finger on the patient's hand 545 to the next collection aperture for saturated the next target spot on the DBS collection card 400, and so on.

The system of FIG. 5 is shown as a collection device 200 and underlying matching DBS card (400 of FIG. 4, but unseen in FIG. 5) with five collection apertures and five corresponding target spots. In other embodiments, the system 500 may include a matching collection device 200 and DBS card 400 having more or fewer apertures and target spots and in different patterns.

FIG. 6 is a close-up diagram view of one target spot 420 of the objective dried blood spot collection card 400 of FIG. 4 properly saturated with capillary blood from a human according to an embodiment of the subject matter disclosed herein. The target spot 420 is shown, as before, surrounded by four (e.g., tetrapartite) voids 415-418 separated by bridges 425 that couple the central target spot 420 to the rest of the DBS card 400. As seen in the shaded area, capillary blood has saturated the central target area 420 and blood has also saturated the bridges 425 as shown by the shaded area extending slightly into the rest of the DBS card 400. In this manner, a visual inspection reveals quickly that enough blood has been collected on the central target disc 420 because of the saturation of the bridges into the DBS card 400.

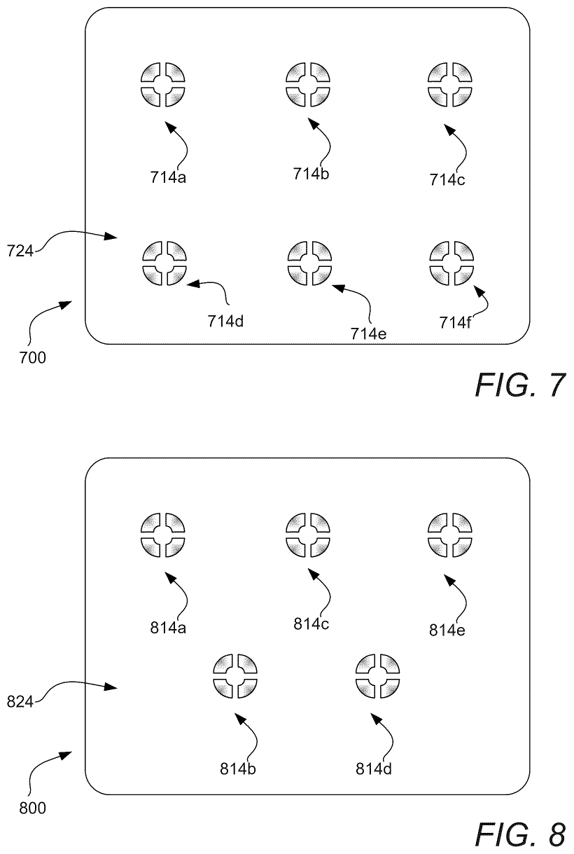

FIG. 7 is a diagram view of another embodiment of an objective dried blood spot collection card 700 according to an embodiment of the subject matter disclosed herein. As before, dried blood spot specimens are collected by applying a few drops of blood, drawn by lancet (e.g., finger prick) from the finger, heel or toe, onto specially manufactured absorbent filter paper. The blood is allowed to thoroughly saturate the filter paper at six specific target circles 714a-f and is air dried thereafter. As can be seen in this embodiment, the six specific target circles 7144a-f are arranged in two rows such that three target circles 714a, 714b, and 714c are disposed in line across an upper portion of the DBS card 700 while three other target circles 714d, 714e, and 714f are disposed in a line across a lower portion of the card 700.

In other embodiments, the number of target spots 714a-f may be more than or less than six. Furthermore, the dimensions of the dried blood spot collection card 700 may be a standard form factor having a length of approximately three inches and a width of five inches, though a skilled artisan understands that this size can be any convenient dimension and the number of spots may be any number including between two and twelve spots. Further, the DBS card 700 is made of thick cellulose cardstock, with 6-mm diameter target discs for blood collection. The inner diameter of the cut-out void sections is 6-mm, while the outer diameter of the voids is 12-mm.

FIG. 8 is a diagram view of yet another embodiment an objective dried blood spot collection card 800 according to an embodiment of the subject matter disclosed herein. As before, dried blood spot specimens are collected by applying a few drops of blood, drawn by lancet (e.g., finger prick) from the finger, heel or toe, onto specially manufactured absorbent filter paper. The blood is allowed to thoroughly saturate the filter paper at five specific target circles 814a-e and is air dried thereafter. As can be seen in this embodiment, the five specific target circles 8144a-e are arranged in a “W” pattern such that three target circles 814a, 814c, and 814e are disposed in line across an upper portion of the DBS card 800 while two other target circles 814b and 814d are disposed in a line across a lower portion of the card 800.

In other embodiments, the number of target spots 814a-e may be more than or less than five. Furthermore, the dimensions of the dried blood spot collection card 800 may be a standard form factor having a length of approximately three inches and a width of five inches, though a skilled artisan understands that this size can be any convenient dimension and the number of spots may be any number including between two and twelve spots. Further, the DBS card 700 is made of thick cellulose cardstock, with 6-mm diameter target discs for blood collection. The inner diameter of the cut-out void sections is 6-mm, while the outer diameter of the voids is 12-mm.

Once dried, specimens may be stored in low gas-permeability plastic bags with desiccant added to reduce humidity, and may be kept at ambient temperature prior to, during, and after shipping. Once in the laboratory, technicians may separate a small disc of saturated paper (e.g., poke a hole) from the sheet using an automated or manual hole punch, dropping the separated disc into a flat-bottomed microtiter plate. The blood is eluted out from the separated disc into a phosphate buffered saline. The resultant plate containing the eluates forms the “master” from which dilutions can be made for subsequent testing.

The use of the terms “a” and “an” and “the” and similar referents in the specification and in the following claims are to be construed to cover both the singular and the plural, unless otherwise indicated herein or clearly contradicted by context. The terms “having,” “including,” “containing” and similar referents in the specification and in the following claims are to be construed as open-ended terms (e.g., meaning “including, but not limited to,”) unless otherwise noted. Recitation of ranges of values herein are merely indented to serve as a shorthand method of referring individually to each separate value inclusively falling within the range, unless otherwise indicated herein, and each separate value is incorporated into the specification as if it were individually recited herein. All methods described herein can be performed in any suitable order unless otherwise indicated herein or clearly contradicted by context. The use of any and all examples, or exemplary language (e.g., “such as”) provided herein, is intended merely to better illuminate embodiments and does not pose a limitation to the scope of the disclosure unless otherwise claimed. No language in the specification should be construed as indicating any non-claimed element as essential to each embodiment of the present disclosure.

Different arrangements of the components depicted in the drawings or described above, as well as components and steps not shown or described are possible. Similarly, some features and sub-combinations are useful and may be employed without reference to other features and sub-combinations. Embodiments have been described for illustrative and not restrictive purposes, and alternative embodiments will become apparent to readers of this patent. Accordingly, the present subject matter is not limited to the embodiments described above or depicted in the drawings, and various embodiments and modifications can be made without departing from the scope of the claims below.

1. A collection card for collecting dried blood spots for diagnostics, comprising:
a card body comprising absorbent filter paper configured to receive one or more blood samples;
a plurality of blood collection targets discs disposed in a pattern on the card body;
a plurality of voids disposed in a pattern surrounding the blood collection targets discs; and
a plurality of bridges disposed between adjacent voids, each bridge in the plurality of bridges coupling one blood collection target disc to the card body.

2. The collection card of claim 1, wherein the plurality of blood collection targets discs comprises six blood collection targets, each blood collection disc comprising a diameter of six millimeters.

3. The collection card of claim 1, wherein the pattern of the plurality of blood collection targets comprises blood collection targets disposed equidistantly in a single line.

4. The collection card of claim 1, wherein pattern of the plurality of blood collection targets comprises blood collection targets disposed in a “W” pattern.

5. The collection card of claim 1, further comprising a form factor configured to placed inside an objective dried blood spot collection device.

6. The collection card of claim 1, further comprising Whatcom™ 903 absorbent paper and a desiccant.

7. The collection card of claim 1, wherein each of the plurality of voids further comprises an elongated crescent shape disposed equidistantly surrounding an adjacent blood collection target disc and forming an overall shape having a diameter of twelve millimeters.

8. The collection card of claim 1, wherein the plurality of voids further comprise a tetrapartite pattern.

9. The collection card of claim 1, wherein the plurality of voids further comprise a tripartite pattern.

10. The collection card of claim 1, wherein the plurality of voids further comprise a quinquepartite pattern.

11. A system for facilitating capillary blood collection, the system comprising:
a dried blood spot collection card, including:
a card body comprising absorbent filter paper configured to receive one or more blood samples;
a plurality of blood collection targets discs disposed in a pattern on the card body;
a plurality of voids disposed in a pattern surrounding the blood collection targets discs; and
a plurality of bridges disposed between adjacent voids, each bridge in the plurality of bridges coupling one blood collection target disc to the card body; and
a dried blood spot collection device, including:
a body having a first portion and second portion coupled to each other and encapsulating a cavity configured to hold the dried blood spot collection card; and
a plurality of blood collection apertures configured to direct blood toward the dried blood spot collection card.

12. The system of claim 11, wherein the plurality of blood collection target spots corresponds to the plurality of blood collection apertures.

13. The system of claim 11, wherein the dried blood spot collection card further comprises five blood spot targets disposed in a “W” pattern with respect to each other and the dried blood spot collection device further comprises five apertures disposed in a “W” pattern.

14. The system of claim 11, wherein the dried blood spot collection card further comprises six blood spot targets disposed in two rows and the dried blood spot collection device further comprises six apertures disposed in two rows.

15. The system of claim 11, further comprising a desiccant.

16. The system of claim 11, wherein the plurality of blood collection targets discs comprises six blood collection targets, each blood collection disc comprising a diameter of six millimeters.

17. The system of claim 11, wherein each of the plurality of voids further comprise an elongated crescent shape disposed equidistantly surrounding an adjacent blood collection target disc and forming an overall shape having a diameter of twelve millimeters.

18. The system of claim 11, wherein the first portion further comprises a plurality of mating pegs disposed on a side of the first portion that is opposite the top face;
wherein the second portion further comprises a plurality of mating receptacles disposed on a side of the second portion that is opposite the bottom face; and
wherein respective mating pegs are configured to mate with respective mating receptacles to removably secure the first portion to the second portion.

19. The system of claim 11, wherein the plurality of voids further comprises a tetrapartite pattern having respective voids separated by at least one bridge.

20. The system of claim 11, further comprising plastic portions having extruded polyurethane with an SPI-A2 rating.
